(19) United States
(12) Patent Application Publication
(10) Pub. No.: US 2022/0409569 A1
(43) Pub. Date: Dec. 29, 2022

(54) Cannabinoid Products with High Bioavailability

(71) Applicant: The Chemist's Cabinet, LLC, Brooklyn, NY (US)

(72) Inventor: Joseph Teglasi, Brooklyn, NY (US)

(21) Appl. No.: 17/772,452

(22) PCT Filed: Oct. 27, 2020
(86) PCT No.: PCT/US2020/057448

§ 371 Date: Apr. 27, 2022

Related U.S. Application Data
(60) Provisional application No. 62/927,238, filed on Oct. 29, 2019.

(51) Int. Cl. A61K 31/352; A61K 47/26; A61K 47/44; A61K 47/24; A61K 31/05

(52) U.S. Cl. CPC A61K 31/352; A61K 47/26; A61K 47/44; A61K 47/24; A61K 31/05

(57) ABSTRACT

Cannabinoids composition with desirable bioavailability of cannabinoids are provided. In some embodiments, the cannabinoid compositions are modular, and thus, they can be divided into smaller portions so that a user may decide precisely how much they will use. In some embodiments, the cannabinoid compositions can be partially or entirely crushed or chewed by the user as a means of increasing the rate of absorption.

### CROSS-REFERENCE TO RELATED APPLICATIONS

The present application is a national stage application of PCT US/2020/057448, filed Oct. 27, 2020, which claims the benefit of the filing date of U.S. provisional patent application Ser. No. 62/927,238, filed Oct. 29, 2019, the entire disclosures of each of the aforementioned applications are incorporated by reference.

### FIELD OF THE INVENTION

The present invention relates to the field of cannabinoids.

### BACKGROUND OF THE INVENTION

Cannabinoids are widely reported as being beneficial for many types of consumers, including consumers of the health, beauty, wellness, and medical industries. In order to capitalize on these benefits, many researchers have been searching for efficient ways to deliver cannabinoids. However, currently well-known technologies suffer from one or more drawbacks.

For example, many researchers are trying to develop ingestible products. One of these types of products is gummies Unfortunately, in commonly used gummies, the cannabinoid content is less than 25 mg per serving or dose, which can be undesirably low.

Because these products are intended to be taken in orally, they can often take several hours to reach peak plasma concentrations. Furthermore, due to the high lipophilicity of cannabinoids and significant degradation by first-pass metabolism, oral administration typically results in a bioavailability of approximately only 6% for cannabidiols (“CBD”), and only 4%-12% for tetrahydrocannabinol (“THC”). Moreover, considering the high retail cost of cannabinoid products that pervades the market, low bioavailability (as well as low cannabinoid concentration and long peak-plasma-concentration-latency) results in not only a waste of cannabinoids, but also both a waste of money and often a lack of any noticeable effect or benefit for the consumer.

Another line of products for delivery of cannabinoids that has been developed includes vape juice and vape pens. One benefit of delivery through these types of vape products is that inhalation results in a significantly higher potential for bioavailability than oral administration, up to 56%, which allows the cannabinoids contained therein to reach peak plasma concentrations in as little as nine minutes. However, bioavailability by inhalation can be highly variable. Each individual inhales a different volume, and holds his or her breath for a different amount of time. Consequently, despite the potential for high bioavailability, in practice, inhalation can result in bioavailability as low as 2%. Furthermore, vaping, like smoking cigarettes, has an associated societal stigma and is not permitted in many locations. Finally, the safety of vaping, in general, is being scrutinized by the medical community.

A third type of products are ones that are harder than gummies, but that are still delivered orally, e.g., hard candies and lozenges. These products are usually administered sublingually, and while the sublingual bioavailability of cannabinoids has not been well-documented, it is estimated to be near the higher end of the gap in bioavailabilities between ordinary oral and inhaled cannabinoid products. The degree of the bioavailability of these products is likely due to the fact that sublingual administration avoids the first-pass effect, resulting in more of the cannabinoids being absorbed and bioavailable. However, known products in these categories still typically have less than 25 mg of cannabinoids per dose, and therefore may produce little effect in persons who have low sensitivity to cannabinoids. Therefore, in order to achieve desired results, users must consume many of these products, resulting in an increased intake of the other ingredients, primarily sugar and sugar alcohols (xylitol, maltitol, etc.) and an undesirably high cost.

Because existing applications for delivery of cannabinoids has significant drawbacks, there remains a need to develop new technologies for delivery of cannabinoids with satisfactory levels of bioavailability.

### SUMMARY OF THE INVENTION

The present invention provides cannabinoid compositions with desirable levels of bioavailability, methods for making these compositions, methods of administering these compositions, and uses of these compositions. Through various embodiments of the present invention, one can efficiently deliver cannabinoids to a subject.

According to a first embodiment, the present invention provides a cannabinoid composition comprising: (a) one or more cannabinoids; (b) erythritol; (c) an oil; and (d) phosphatidylcholine. A “cannabinoid composition” may be a mixture, matrix, solution, or other combination of two or more ingredients, at least one of which is a cannabinoid. Furthermore, a cannabinoid composition may be a product or a formulation that contains one or more cannabinoids and one or more other ingredients in which the cannabinoids are dissolved, housed, stored, suspended, or with which they are otherwise associated. The identity and amount of these other ingredients may impact desirable bioavailability profiles of the cannabinoid molecules.

According to a second embodiment, the present invention provides a method for preparing a cannabinoid composition. The method comprises: (a) preparing a cannabinoid mixture at a mixing temperature, wherein said cannabinoid mixture comprises a cannabinoid source, an oil, phosphatidylcholine, and a flavoring, wherein the cannabinoid source comprises, consists essentially of or consists of a set of one or more cannabinoids, each of the one or more cannabinoids has a degradation temperature, and the mixing temperature is lower than the degradation temperature of each of the one or more cannabinoids; (b) preparing an erythritol mixture, wherein the erythritol mixture comprises, consists essentially of or consists of erythritol, and optionally, a sugar source, an acid, and water; (c) heating the erythritol mixture to a temperature of at least 250° F. to form a sugar matrix; (d) cooling the sugar matrix for up to 120 seconds; (e) mixing the cannabinoid mixture with the sugar matrix to form a cannabinoid-charged matrix; and (f) transferring the cannabinoid-charged matrix into a mold to form a cannabinoid composition.

According to a third embodiment, the present invention provides another method for preparing a cannabinoid composition. This method comprises: (a) preparing a cannabinoid mixture at a mixing temperature, wherein said cannabinoid mixture comprises a cannabinoid source, an oil, phosphatidylcholine, and a flavoring, wherein the cannabinoid source comprises, consists essentially of or consists of a set of one or more cannabinoids, each of the one or more cannabinoids has a degradation temperature and the mixing temperature is lower than the degradation temperature of each of the one or more cannabinoids; (b) preparing an erythritol mixture, wherein said erythritol mixture comprises, consists essentially of or consists of erythritol, and optionally, a sugar source, an acid, and water; (c) heating the erythritol mixture to a temperature of at least 250° F. to form a sugar matrix; (d) cooling the sugar matrix for up to 120 seconds; (e) mixing the cannabinoid mixture with the sugar matrix to form a cannabinoid-charged matrix; and (f) transferring the cannabinoid-charged matrix onto a flat surface to form a cannabinoid composition.

According to a fourth embodiment, the present invention provides another method for preparing a cannabinoid composition. This method comprises: (a) preparing a cannabinoid mixture at a mixing temperature, wherein said cannabinoid mixture comprises, consists essentially of or consists of a cannabinoid source, an oil, phosphatidylcholine, and a flavoring, wherein the cannabinoid source comprises a set of one or more cannabinoids, each of the one or more cannabinoids has a degradation temperature, and the mixing temperature is lower than the degradation temperature of each of the one or more cannabinoids; (b) preparing an erythritol mixture, wherein the erythritol mixture comprises, consists essentially of or consists of erythritol, and optionally, a sugar source, an acid, and water; (c) heating the erythritol mixture to a temperature of at least 250° F. to form a sugar matrix; (d) cooling the sugar matrix for up to 120 seconds; (e) mixing the cannabinoid mixture with the sugar matrix to form a cannabinoid-charged matrix; (f) transferring the cannabinoid-charged matrix into a container; (g) cooling the cannabinoid-charged matrix while said cannabinoid-charged matrix is in said container to form a solidified cannabinoid composition; and (h) rolling, pulling and die cutting the solidified cannabinoid composition.

In various embodiments, one or more of the following benefits can be realized: desirable bioavailability, relatively high potency, avoidance of undesirable levels of side-effects, modularity in that compositions can easily be divided into desirable portions or dosages; an ability to be crushed or chewed by a user; and a desirably long shelf-life.

### BRIEF DESCRIPTION OF THE FIGURES

FIG. 1 is a flow chart of certain methods of the present invention.

FIG. 2 is a representation of a micelle of some embodiments of the present invention.

FIG. 3 is a representation of movement cannabinoids of the present invention into the bloodstream of a subject.

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided herein for the convenience of the reader and do not limit the scope of any of the embodiments disclosed herein.

Cannabinoid Compositions

According to a first embodiment, the present invention provides a cannabinoid composition comprised of: (a) one or more cannabinoids; (b) erythritol; (c) an oil; and (d) phosphatidylcholine. The cannabinoid composition may be a mixture, solution, suspension, or other combination of ingredients, and the cannabinoid composition may also be referred to as a formulation or cannabinoid containing product or cannabinoid product.

Cannabinoids

The cannabinoid compositions comprise one or more cannabinoids. The one or more cannabinoids within a cannabinoid composition may be referred to as a “cannabinoid source” and includes but is not limited to one or more cannabinoids that have been purified or otherwise processed or extracted from their natural state, or are in their raw forms, i.e., unprocessed, or are partially processed, and thus contain one or more impurities, or combinations thereof. Additionally, some, none, or all of the cannabinoids may be synthetic.

A cannabinoid is defined as any molecule naturally found in the cannabis plant that can activate the CB1 (cannabinoid receptor type 1) and/or CB2 (cannabinoid receptor type 2) receptors and/or are or have structural similarities to THC, e.g., any terpene or terpenoid naturally found in the cannabis plant. Non-limiting examples of cannabinoids are cannabidiol (CBD), tetrahydrocannabinol (THC), dronabinol, nabilone, cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM). Unless otherwise provided, within the scope of a cannabinoid is a salt thereof.

Because each cannabinoid composition may contain more than one type of cannabinoid molecule, one may refer to a composition having a “total cannabinoid content.” The total cannabinoid content refers to the total amount of cannabinoids, regardless of the identity of any particular one or more cannabinoid molecules.

In some embodiments, the total cannabinoid content comprises, consists essentially of, or consists of CBD or THC or combinations thereof. In some cannabinoid compositions, the total cannabinoid content is 100% CBD or 100% THC, or a combination of CBD and THC in which there are equal amount of each of CBD and THC or more CBD than THC or more THC than CBD. For example, the CBD to THC ratio may be 1:10 to 10:1 or 1:5 to 5:1 or 1:3 to 3:1 or 1:2 to 2:1 or approximately 1:1. In some embodiments, there is an absence of any cannabinoid other than THC, or other than CBD, or other than THC and CBD, or there is a de minimis amount of other cannabinoids, e.g., less than 5 wt. %, less than 4 wt. %, less than 3 wt. %, less than 2 wt. %, less than 1 wt. %, less than 0.1 wt. % of other cannabinoids based on the total cannabinoid content.

The cannabinoids may come from natural sources or be synthetic. When from a natural source, the cannabinoid source may, for example, come from an extract of a cannabis plant, which may be a hemp plant or a marijuana plant or a combination of extracts from one or both of these types of plants.

In some embodiments, within the total cannabinoid content, the amount of THC is less than or equal to 0.3%. This level of THC is commonly found in industrial hemp, e.g., a wax, shatter, or crumble. In other embodiments, the total cannabinoid content is greater than 0.3% THC. This level of THC is commonly found in marijuana.

In some embodiments, the one or more cannabinoids have a total cannabinoid content of between 0.5% and 10% or between 1% and 10% or between 1% and 5% or between 2 and 5% by weight based on the weight of the cannabinoid composition.

Another measure for the total cannabinoid composition is based on absolute weight. In some embodiments, the total cannabinoid content is at least 25 mg, at least 30 mg, at least 35 mg, at least 40 mg, or at least 50 mg. Further, because in some embodiments, the combined composition is structured to be modular, e.g., a bar that one can break into uniform units, each unit may have a total cannabinoid content of at least 25 mg, at least 30 mg, at least 35 mg, at least 40 mg, or at least 50 mg, and the bar may, for example, have 2, 4, 6, 8, 10, 12, 14, 16, 18, or 20 units.

Erythritol

Erythritol is a sugar alcohol with the following structure:

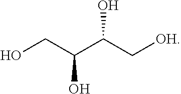

It may be found naturally in some fruits and fermented foods. Additionally, it can be produced from glucose by fermentation with yeast. A few of the benefits of erythritol are that although it is sweet, it is low in calories, it generally does not affect one's blood sugar, it does not cause tooth decay, and it can be absorbed by the body before it reaches the intestine and is excreted by the body. The ability of the body to absorb erythritol before it reaches the intestine prevents the erythritol from killing off gut flora. Consequently, it may cause significantly less GI upset than other sugar alcohols.

Furthermore, erythritol has a positive heat of solvation and is consequently non-hygroscopic. This positive heat of solvation of erythritol causes the cannabinoid compositions of the present invention to dissolve slower than products produced with other sugar alcohols, which facilitates the delivery of the cannabinoids.

In some embodiments, the erythritol is present in an amount of about 10%-99% or 15%-85% or 20%-80% or 25%-75% or 30%-70% or 35%-65% or 40%-60% by weight based on the weight of the cannabinoid composition.

Within the scope of the present invention are the use of not only erythritol but also derivatives thereof.

Oils

The compositions of the present invention may contain one or more oils. By way of non-limiting examples, the oil may consist of, consist essentially of, or comprise sesame oil, canola oil, or a combination of sesame oil and canola oil. When using a combination of sesame oil and canola oil, the combination may, for example, be in a ratio of about 1:10 to 10:1 or 1:5 to 5:1 or 1:3 to 3:1 or 1:2 to 2:1 or approximately 1:1 sesame oil to canola oil based on the weight or volume of the respective oils.

In some embodiments, the total oil content oil makes up about 0.1 wt. %-5 wt. %; 1 wt. %-5 wt. %; 1 wt. %-5 wt. %; 1 wt. %-4 wt. %; or 2 wt. %-3 wt. % of the cannabinoid composition.

Phosphatidylcholine

Phosphatidylcholine refers to a class of phospholipids that contain choline, glycerophosphoric acid and two fatty acids that may be the same or different. It may be represented by:

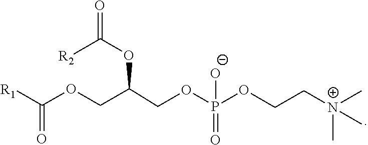

R₁ and R₂ are aliphatic hydrocarbon chains, e.g., oleyl or palmityl groups. In some embodiments, the phosphatidylcholine makes up about 0.1 wt. %-2 wt. %; 0.5 wt. %-2 wt. %; or 1 wt. %-2 wt. % of the cannabinoid composition. Each of R₁ and R₂ may independently be substituted or unsubstituted; branched, cyclic, or linear; and in some embodiments contain 1 to 30 or 2 to 26 or 8 to 24 or 10 to 20 carbons. Additionally, each of R₁ and R₂ may independently be saturated or mono- or polyunsaturated, and may or may not have aromatic substituents. Within a cannabinoid composition, there may be a single type of phosphatidylcholine or a combination of phosphatidylcholines.

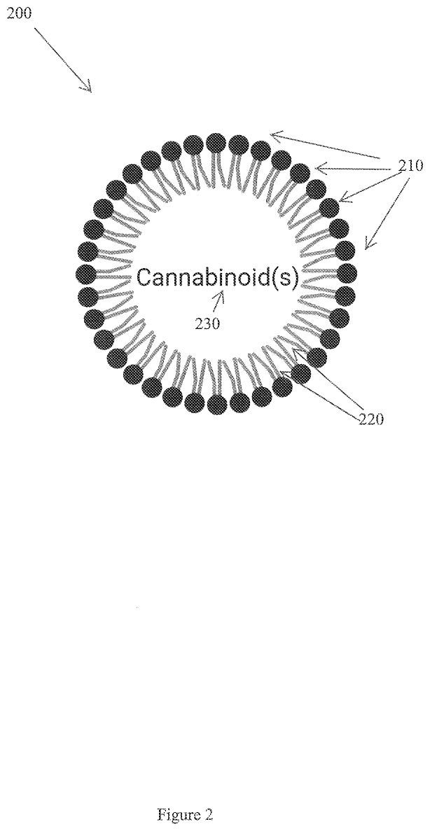

In some embodiments, the one or more cannabinoids and the phosphatidylcholine form one or more clusters, wherein each cluster has a hydrophilic region and a lipophilic region. Further, in some embodiments, at least one of the one or more clusters, e.g., at least 25%, at least 50%, at least 75%, or at least 90%, is a micelle or a plurality of micelles. An example of a micelle is shown in FIG. 2. Within the micelle 200, the hydrophilic region 210 may be an exterior region and the lipophilic region may be an interior region 220. Additionally, within the micelle, there may be the cannabinoid(s) 230.

Sugars

In some embodiments, the cannabinoid composition also comprises one or more sugars or derivatives thereof. The one or more sugars may comprise, consist essentially of, or consist of simple sugars (monosaccharides) such as glucose, galactose, or fructose; disaccharides such as sucrose, maltose, or lactose; or polysaccharides such as starches or combinations thereof. The sugar may be derived from any source (a “sugar source”), which by way of a non-limiting example is an edible sugar source such as honey or cane juice. One way to derive a sugar is through evaporation. Thus, in some embodiments, the sugar may be added as evaporated cane juice.

In some embodiments, when sugar is present, it may be present in an amount that is up to about 50 wt. % of the cannabinoid composition. Thus, in some embodiments, there may be sugar in an amount of 0 wt. %-50 wt. %; 0.01 wt. %-50 wt. %; 0.1 wt. %-50 wt. %; 1 wt. %-50 wt. %; 1 wt. %-40 wt. %; 1 wt. %-40 wt. %; 5 wt. %-40 wt. %; 5 wt. %-35 wt. %; 10 wt. %-35 wt. %; or 10 wt. %-25 wt. %.

In alternative embodiments in which one wishes to add sweetness to the cannabinoid composition, one may, in whole or in part, use a sugar substitute such as saccharin, aspartame, or stevia. When a sugar substitute is used alone, it may be used in the quantities described above in which sugars may be used. When sugar substitutes are to be used in combination with sugar, the combined amount of the sugar and the sugar substitute may be within the ranges described above for quantities of sugar.

Flavorings

In some embodiments, the cannabinoid composition also comprises a flavoring. Erythritol itself may be considered a flavoring, and in some embodiments erythritol is the only flavoring while in other embodiments, the cannabinoid composition comprises a flavoring other that erythritol. In some embodiments, when a flavoring is present, it may be present in an amount of 0.1 wt. %-5 wt. %; 1 wt. %-5 wt. %; 1 wt. %-4 wt. %; or 2 wt. % to 3 wt. % based on the weight of the cannabinoid composition.

Examples of flavorings include, but are not limited to, maple, vanilla, chocolate, cinnamon, and fruit flavorings such as monk fruit, raspberry, strawberry, blueberry, and pineapple.

Acids

In some embodiments, the cannabinoid composition also comprises one or more acids. Each acid may be one that is not harmful when ingested, e.g., an acid from a fruit. Thus, an acid may be in the form of citrus juice, lemon juice, pineapple juice, lime juice, or orange juice, or derived or extracted form one or more of these juices.

In some embodiments, when an acid is present, it may be present in an amount of 0.1 wt. %-6 wt. %; 1 wt. %-6 wt. %; 1 wt. %-5 wt. %; 1 wt. %-4 wt. %; or 2 wt. %-3 wt. % based on the weight of the cannabinoid composition.

In some embodiments, the oil described above can serve the function of an acid and optionally remove the need for an additional acid. For example, one may use a long or medium-chain fatty acid that is also an oil.

Water

In some embodiments, the cannabinoid composition also comprises water. In some embodiments, when water is present, it may be present in an amount of 0.1 wt. %-6 wt. %; 1 wt. %-6 wt. %; 1 wt. %-5 wt. %; 1 wt. %-4 wt. %; or 2 wt. %-3 wt. % based on the weight of the cannabinoid composition.

Food Coloring Agents

In some embodiments, the cannabinoid composition also comprises one or more food coloring agents, e.g., one or more natural food coloring agents. In some embodiments, when food coloring is present, it may be present in an amount of 0.1 wt. %-5 wt. %; 1 wt. %-5 wt. %; 1 wt. %-5 wt. %; 1 wt. %-4 wt. %; or 2 wt. %-3 wt. %. Examples of natural food coloring agents include but are not limited to carotenoids, chlorophyllins, anthocyanins, betanin, annatto, caramel coloring, carmine, elderbery juice, lycopene, paprika, and turmeric. Examples of synthetic food coloring agents include but are not limited to FD&C Blue No. 1; FD&C Blue No. 2; FD&C Green No. 3; FD&C Red. No. 3, FD&C Red No. 40, FD&C Yellow No. 5, and FD&C Yellow No. 6.

Table I provides an example of the ranges of various components of the cannabinoid compositions of some embodiments of the present invention:

TABLE I

| Component | Amount (wt. %) |
|---|---|
| Erythritol | 10%-99% |
| Sugar Source(s) | 0%-50% |
| Flavoring | 0.1%-5% |
| Oil | 0.1%-5% |
| Phosphatidyl Choline | 0.1%-2% |
| Lemon Juice | 0.1%-6% |
| Water | 0.1%-6% |
| Cannabinoid Source(s) | 0.5-10% |
| Food Coloring | 0%-5% |

Matrices

In some embodiments of the present invention, within the cannabinoid compositions, the clusters of the one or more cannabinoids and phosphatidylcholine are within a matrix. By way of a non-limiting example, the cannabinoid composition may comprise: (a) one or more cannabinoids; (b) erythritol; (c) an oil; (d) phosphatidylcholine; (e) sugar; and (f) water, wherein the one or more cannabinoids and the phosphatidylcholine form a plurality of clusters. Each cluster may have a hydrophilic region and a lipophilic region, and the plurality of clusters may be dispersed within a matrix comprising erythritol, sugar, and water. In some of these embodiments, one or more of the clusters is in the form of a micelle.

Without being bound by any one theory, in some embodiments, a small particle comprising, consisting essentially of, or consisting of one or more cannabinoid molecules undergoes a lipophilic interaction with the aliphatic chains on phosphatidylcholine molecules, resulting in the formation of a micelle or cluster with a lipophilic cavity and a hydrophilic exterior, which allows the micelles/clusters/aggregates to be dispersed regularly or irregularly throughout a matrix that consists primarily of more hydrophilic molecules, such as erythritol, sugars (e.g., from honey) and water.

Methods of Preparation

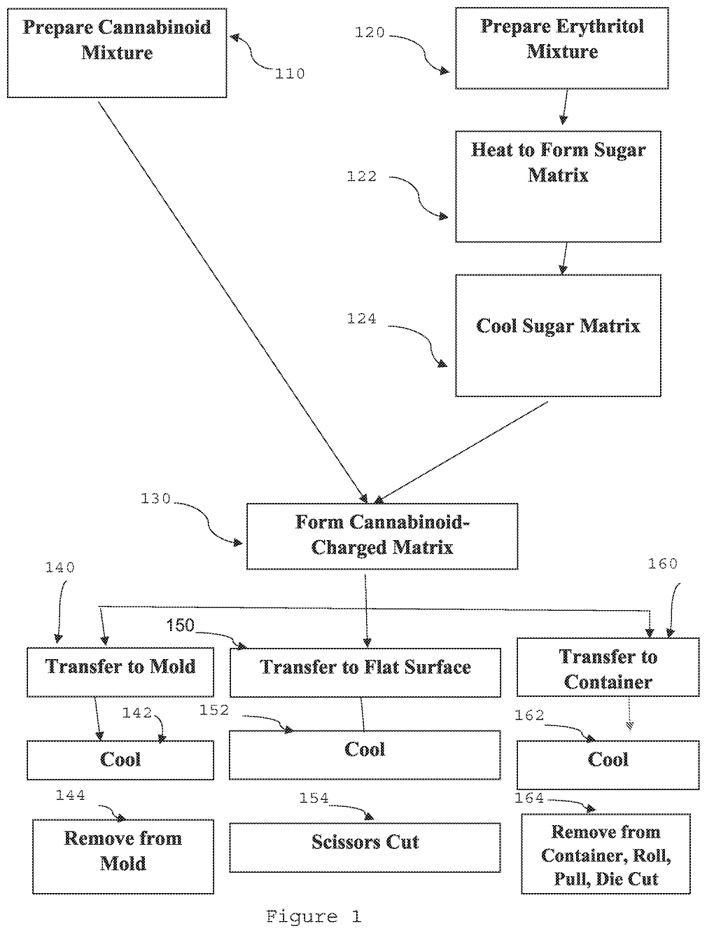

In some embodiments, the present invention is directed to methods for preparing a cannabinoid composition. FIG. 1 provides an example of some of the methods of the present invention for preparing a cannabinoid composition. As shown in that figure, one may begin with preparing a cannabinoid mixture 110 at a mixing temperature so that the cannabinoid mixture comprises a cannabinoid source, an oil, phosphatidylcholine, and a flavoring. The cannabinoid source may comprise a set of one or more cannabinoids. Each of the one or more cannabinoids has a degradation temperature, and the mixing temperature is lower than the degradation temperature of each of the one or more cannabinoids, i.e., lower than the lowest degradation temperature of any cannabinoid in the cannabinoid mixture.

The cannabinoid source may, for example, comprise CBD, THC or a combination thereof in the ratios described above, and it may be all natural, all synthetic or a combination thereof. One cannabinoid source that may be advantageous in some embodiments is industrial hemp. The industrial hemp may, for example, be in the form of a wax, a shatter, or a crumble. Another cannabinoid source that may be advantageous in some embodiments is a cannabis plant or an extract of a cannabis plant.

These methods may also comprise preparing an erythritol mixture 120, which may be formed before, after, or at the same time as the cannabinoid mixture. The erythritol mixture comprises erythritol, a sugar source, an acid, and water. Next one heats the erythritol mixture to a temperature of at least 250° F. to form a sugar matrix. In some embodiments, the heating is to a temperature of 260° F. to 350° F. or 270° F. to 340° F. or 280° F. to 330° F. or 290° F. to 320° F. or 300° F. to 310° F. Heating of the erythritol mixture may allow the formation of the sugar matrix 122, which may be a matrix of the sugar from the sugar source and erythritol. Thus, the sugar matrix is formed by the combination of erythritol and sugars, if present, with acid and water. The heating of the matrix causes the evaporation of the majority of the water. At the same time, some of the sugar/sugar alcohol molecules oligomerize. This increases inter-molecular interactions, primarily through hydrogen bonding.

The sugar matrix is next cooled 124. The cooling time may, for example, be up to 120 seconds, up to 90 seconds, up to 75 seconds, up to 60 seconds, or up to 45 seconds or up to 30 seconds. Cooling may be accomplished by removing the heat, or optionally, by placing the matrix in an environment that is cooler than the heat source. Because of the inter-molecular interactions that were formed, upon cooling, a strong crystal structure can be created.

The cannabinoid mixture is next mixed with the sugar matrix to form a cannabinoid-charged matrix 130. Mixing may, for example, be by one or both of stirring and agitation of a container containing the cannabinoid mixture and sugar matrix. In some embodiments, the mixing is over a period of 30 seconds or less or 20 seconds or less or 15 seconds or less. The mixing step may overlap in part or in whole with the cooling step or it may be after the cooling step, e.g., shortly (a few seconds up to two minutes) after the cooling step.

By way of example, in FIG. 1 three non-limiting options are shown for the cannabinoid-charged matrix. The particular path that one selects will depend on the desired product. In a first option, after the cannabinoid-charged matrix is formed, it is transferred into a mold 140 to form a cannabinoid composition. Molds made be designed and selected to have the desired aesthetic e.g., a desired shape. Optionally, a mold may also contain one more desired shapes or symbols on the surface such as brand names or trademarks. The molds may also be designed to generate products that are breakable into smaller units and thus are modular. For example, repeating shapes of a first depth may be connected by regions of a second depth that lend themselves to easy breaking apart after removal from the mold at the locations in the product that corresponds to the second depth.

The transfer to the mold may, for example, be by pouring. This pouring step may be over a long or short period of time. In some embodiments the pouring takes place over a period of time that is less than or equal to 120 seconds, less than or equal to 90 seconds, less than or equal to 75 seconds, less than or equal to 60 seconds, less than or equal to 45 seconds, or less than or equal to 30 seconds. The transfer may be initiated immediately after mixing or after a small amount of time after mixing, e.g., up to 2 minutes, up to 1 minute, or up to 30 seconds.

In some embodiments, after the cannabinoid-charged matrix is transferred into the mold, the mold and its contents are cooled 142. This cooling may be under conditions that allow for crystallization within the composition. To allow for optimal crystallization, it may be advantageous to refrain from moving the mold for a period of at least 15 seconds, or at least 30 seconds, or at least 45 seconds, or at least 60 seconds, or at least 120 seconds.

After the mold has been cooled, and optionally the desired crystallization has occurred, the composition may be removed 144 from the mold and stored, or it may be stored within the mold. When storing the composition, in some embodiments, storage may be under conditions in which there is an absence of light of wavelengths of less than 400 nm; under conditions in which there is an absence of light of wavelengths of less than 500 nm; under conditions in which there is an absence of light of wavelengths of less than 600 nm; under conditions in which there is an absence of light of wavelengths of less than 700 nm; or under conditions in which there is an absence of light in the visible spectrum.

Some compositions may be stored in a colored glass vessel. Examples of colored glass vessels that may be of use include, but are not limited to, those that are brown or amber in color.

In some embodiments, rather than transferring the cannabinoid-charged matrix to a mold, one pours it onto a flat surface 150. By pouring the cannabinoid-charged matrix onto a flat surface, the resulting composition will be thinner than if put into a mold that has any measurable depth. This cannabinoid-charged matrix may be cooled 152 and/or allowed to crystallize, and subsequently cut 154. By pouring the cannabinoid-charged matrix onto a flat surface, the resulting composition will be thinner than if put into a mold that has any measurable depth.

In some embodiments, rather than transferring the cannabinoid-charged matrix to a mold or pouring it onto a flat surface, one puts the cannabinoid-charged matrix in a container 160; cools the cannabinoid-charged matrix while said cannabinoid-charged matrix is in said container 162 to form a solidified cannabinoid composition; and rolls, pulls and die cuts the solidified 164 cannabinoid composition. This process may be particularly advantageous when the ultimate product is to be chewable such as one that is in the form of a gummies or other soft and chewable items such as a taffy that contains the cannabinoid composition.

Dosing Forms

The cannabinoid compositions of the present invention may be in any one or more of a number of forms. For example, they may be in the form of tablets, capsules, creams, ointments, injectables, or powders. Further, they may be incorporated into items, such as hard candy or soft chewable candies, and beverages, such as juices or combined with active ingredients of pharmaceuticals, vitamins, supplements or nutraceuticals in the form of gummies, lozenges, or dissolvable products. Still further, they may be incorporated into cosmetics. They may also be contained within vape juice or vape pens.

In some embodiments, the cannabinoid compositions are in the form of a powder. Accordingly, after the cannabinoid composition is formed, it may, for example, be crushed by a mechanical force and/or sonication. This powder may then be combined with other active or other inactive ingredients in any of the aforementioned products.

In some embodiments, the compositions are modular. A composition is modular if it can easily be cut or broken into smaller portions. The modularity allows for greater cost-effectiveness for consumers who are sensitive to cannabinoids.

In some embodiments, e.g., the lozenges or gummies, the formulation can be chewed to increase the rate of absorption. As persons of ordinary skill in the art will recognize, the texture and chewability of gummies, such as gummy bears, is due to its ingredients. Common ingredients for gummies are sugar, glucose syrup, starch, flavoring, food coloring, citric acid, and gelatin, any one or more of which may be mixed or otherwise incorporated into the products of the present invention.

Administration

In some embodiments, the present invention provides a method for administering a cannabinoid composition. Administration may be for purposes of treating, preventing, or managing a condition, a disease, or a disorder in a subject. Examples of uses of the cannabinoid composition are for the treatment, prevention, or management of pain, inflammation, anxiety, blood pressure, depression, seizures, mental health, acne, Parkinson's disease, glaucoma, ulcerative colitis, somnolence, or insomnia. Thus, in some embodiments, the present invention is directed to a method of treating, preventing or managing pain, inflammation, anxiety, blood pressure, depression, seizures, mental health, acne, Parkinson's disease, glaucoma, ulcerative colitis, somnolence, or insomnia, said method comprising administering a cannabinoid composition of the present invention to a subject in need thereof.

As used herein, unless otherwise specified, the terms “treat,” “treating,” and “treatment” refer to alleviating or abrogating a disease, or one or more of the symptoms associated with the disease; or alleviating or eradicating the cause(s) of the disease itself.

As used herein, unless otherwise specified, the term “preventing” refers to the treatment with or administration of a compound provided herein, with or without another additional active compound, prior to the onset of symptoms, particularly to subjects at risk of a disease or disorder described herein. The term “prevention” includes the inhibition or reduction of a symptom of the particular disease. Subjects with familial history of a disease in particular are candidates for preventive regimens in certain embodiments. In addition, subjects who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. In certain cases, the beneficial effects that a subject derives from a prophylactic agent do not result in a cure of the disease or disorder. In certain cases, the beneficial effects that a subject derives from a therapeutic agent do not result in a cure of the disease or disorder.

Similarly, in some embodiments, the present invention is directed to a medicament comprising a cannabinoid composition that can be administered to a subject in need thereof. The need may, for example, be any one or more of the aforementioned conditions.

Examples of methods of administration include but are not limited to orally, buccally, sublingually, rectally, or vaginally. When administered orally, the product may be broken or chewed into pieces and swallowed. Chewing more thoroughly may increase the rate of delivery. When administered buccally, the product may be held either sublingually or against any part of the tongue or oral mucosa. To increase the rate of delivery (and usually at the expense of total bioavailability), the product may be chewed to the desired extent. Completely chewing and holding the powder under the tongue (or against any other part of the oral mucosa with a large surface area) results in the fastest rate of absorption (as well as the most product swallowed in the saliva-phase).

When administered rectally, e.g., as part of a suppository, it may be advantageous to use a mold that causes the shape of the composition to allow for easy insertion in a way that is similar to how currently marketed suppositories are shaped.

When administered vaginally, it may be advantageous to use a mold that causes the shape of the composition to allow for easy insertion into the vaginal canal and at a desirable depth.

The subject may be a human or the subject may be a non-human animal. Examples of non-human animals are other mammals such as other primates, mice, rats, cats, dogs, horses, sheep, cows, and pigs.

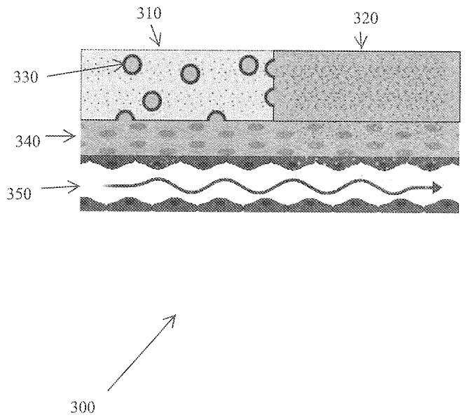

In some embodiments, the cannabinoids of the cannabinoid compositions of the present invention may be accessible to a subject through one or a combination of two pathways that are illustrated in FIG. 3. In one pathway there is solid/mucosa direct diffusion. In this method, a solid matrix 320 may be held against the mucosa 340, and the lipophilic components (including the cannabinoids) dissolve directly into the mucosa along the solid/mucosa interface. Within the solid phase of the figure, cannabinoid molecules are shown free as small dots. They may dissolve directly into the phospholipid bilayer of the epithelial cell membranes.

In a second mechanism, there is indirect diffusion. The solid matrix may be dissolved in water, or saliva 310, which results in the formation of micelles 330 that are similar to the micelles or clusters that are used to evenly distribute the cannabinoids within the matrix during the production process. These micelles or clusters then diffuse throughout the volume of the solution 340. During the course of diffusion, the micelles and/or clusters collide with the mucosa, and sometimes dissolve into it.

These two aforementioned pathways converge. The mucosa 340 becomes concentrated with the cannabinoid(s) and the cannabinoid(s) diffuse along the concentration gradient into nearby capillaries 350. From there, the cannabinoid(s) are delivered to all tissues of the body via the circulatory system.

The relative contribution of each pathway is determined by the relative rates of diffusion of the solid into the mucosa vs. into the solution. The rate of the former is controlled by the surface area of the solid/mucosa interface, and the rate of the latter is controlled by the surface area of the solid/solution interface. Note that when the mucosa is the oral mucosa, the latter pathway results in more cannabinoids getting swallowed, and therefore reduces the overall bioavailability of the product. Therefore, a degree of chewing a solid matrix into smaller pieces increases the surface area of both interfaces to that degree, and so results in a corresponding increase in the rate of absorption, as well as a corresponding reduction in the quantity of cannabinoids that are absorbed.

1. A cannabinoid composition comprising:
(A) one or more cannabinoids;
(B) erythritol;
(C) an oil; and
(D) phosphatidylcholine.

2. The composition of claim 1, wherein the oil is sesame oil.

3. The composition of claim 1, wherein the oil is canola oil.

4. The composition of claim 1 further comprising a sugar.

5. The composition of claim 4, wherein the sugar is derived from honey.

6. The composition of claim 4, wherein the sugar is derived from cane juice.

7. The composition of claim 6 further comprising a flavoring, wherein the flavoring is not erythritol.

8. The composition of claim 7 further comprising an acid.

9. The composition of claim 8, wherein the acid is lemon juice.

10. The composition of claim 7 further comprising water.

11. The composition of claim 10 further comprising a food coloring agent.

12. The composition of claim 1, wherein the phosphatidylcholine has the formula: 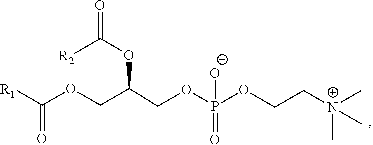
wherein R₁ and R₂ each contain 1 to 30 carbons and is saturated or mono-or polyunsaturated.

13. The method of claim 13, wherein R₁ and R₂ each contain 8 to 24 carbons.

14. The method of claim 13, wherein each of R₁ and R₂ is an oleyl or palmityl group.

15. The method of claim 13, wherein either or both of R₁ and R₂ has at least one aromatic substituent.

16. The composition of claim 1, wherein the erythritol is present in an amount of 10%-99% by weight based on the weight of the composition.

17. The composition of claim 1 further comprising an acid.

18. The composition of claim 17, wherein the acid is lemon juice.

19. The composition of claim 1 further comprising a food coloring agent.

20. The composition of claim 19, wherein the food coloring agent is a natural food coloring agent.

21-74. (canceled)
